(19) United States
(12) Patent Application Publication
(10) Pub. No.: US 2017/0167999 A1
(43) Pub. Date: Jun. 15, 2017

(54) SEMICONDUCTOR TYPE GAS SENSOR, METHOD OF MANUFACTURING SEMICONDUCTOR TYPE GAS SENSOR, AND SENSOR NETWORK SYSTEM

(71) Applicant: ROHM CO., LTD., Kyoto (JP)

(72) Inventor: Shunsuke AKASAKA, Kyoto (JP)

(73) Assignee: ROHM CO., LTD., Kyoto (JP)

(21) Appl. No.: 15/372,871

(22) Filed: Dec. 8, 2016

(30) Foreign Application Priority Data
Dec. 11, 2015 (JP) 2015-242090

(51) Int. Cl. G01N 27/12; B81C 1/00; G01N 33/00; B81B 7/00

(52) U.S. Cl. CPC G01N 27/125; B81B 7/0061; B81C 1/00206; G01N 33/004; B81B 2203/04; B81C 2201/0132; B81C 2201/0176

(57) ABSTRACT

A semiconductor type gas sensor for detecting a CO₂ gas includes: a gas-sensitive body in which a surface of a tin oxide is coated with a thin film of a rare earth oxide; a pair of positive and negative electrodes tightly formed on the gas-sensitive body; and a micro-heater configured to heat the gas-sensitive body.

### CROSS-REFERENCE TO RELATED APPLICATION(S)

This application is based upon and claims the benefit of priority from Japanese Patent Application No. 2015-242090, filed on Dec. 11, 2015, the entire contents of which are incorporated herein by reference.

### TECHNICAL FIELD

The present disclosure relates to a semiconductor type gas sensor, a method of manufacturing a semiconductor type gas sensor, and a sensor network system.

### BACKGROUND

Recently, a demand for CO₂ gas sensors for measuring a concentration of a carbon dioxide (CO₂) in the air has increased. In such CO₂ gas sensors, a CO₂ gas sensor based on infrared spectroscopy using infrared absorption of CO₂ is the mainstream. Recently, a semiconductor type CO₂ gas sensor for measuring a concentration of CO₂ using a gas-sensitive body having tin oxide (snO₂) as a main ingredient is also known.

However, using such gas-sensitive body having SnO₂ has a problem of reacting to various gases such as H₂ and CO. Therefore, the semiconductor type CO₂ gas sensor has not become prevalent. It is known in the related art that “sensitivity to carbon dioxide that cannot be generally obtained is enhanced using a La-added tin oxide”, but it is required to further enhance the selectivity of a CO₂ gas.

### SUMMARY

The present disclosure provides some embodiments of a semiconductor type gas sensor capable of further enhancing selectivity of a CO₂ gas, a method of manufacturing a semiconductor type gas sensor, and a sensor network system.

According to one embodiment of the present disclosure, there is provided a semiconductor type gas sensor for detecting a CO₂ gas, including: a gas-sensitive body in which a surface of a tin oxide is coated with a thin film of a rare earth oxide; a pair of positive and negative electrodes tightly formed on the gas-sensitive body; and a micro-heater configured to heat the gas-sensitive body.

According to another embodiment of the present disclosure, there is provided a method of manufacturing a semiconductor type gas sensor for detecting a CO₂ gas, including: forming a micro-heater; forming a pair of positive and negative electrodes on the micro-heater; and tightly forming a gas-sensitive body in which a surface of a tin oxide is coated with a thin film of a rare earth oxide, between the pair of positive and negative electrodes.

According to still another embodiment of the present disclosure, there is provided a sensor network system including the aforementioned semiconductor type gas sensor.

### BRIEF DESCRIPTION OF THE DRAWINGS

FIG. 1 is a schematic structure diagram illustrating a detection circuit of a CO₂ gas sensor according to a first embodiment of the present disclosure.

FIG. 2 is a schematic structure diagram illustrating a CO₂ gas sensor according to a comparative example.

FIG. 3 is a schematic structure diagram illustrating the CO₂ gas sensor according to the first embodiment.

FIGS. 4A and 4B are explanatory views illustrating a principle of detecting a CO₂ gas by the CO₂ gas sensor illustrated in FIG. 3, in which FIG. 4A is a schematic structure diagram of two adjacent SnO₂ grains and FIG. 4B is a graph schematically illustrating conduction bands of the SnO₂ grains.

FIGS. 5A and 5B are schematic structure diagrams illustrating a method of manufacturing a CO₂ gas sensor according to the first embodiment.

FIGS. 6A to 6C are schematic structure diagrams illustrating modifications of a layout of electrodes of the CO₂ gas sensor according to the first embodiment, in which FIG. 6A illustrates the same layout of electrodes as that of FIG. 3, FIG. 6B is modification 1 of the layout of electrodes illustrated in FIG. 3, and FIG. 6C is modification 2 of the layout of electrodes illustrated in FIG. 3.

FIG. 7 is a schematic structure diagram illustrating a CO₂ gas sensor according to a second embodiment of the present disclosure.

FIG. 8 is an explanatory view illustrating a principle of detecting a CO₂ gas by the CO₂ gas sensor illustrated in FIG. 7.

FIGS. 9A and 9B are schematic structure diagrams illustrating a method of manufacturing a CO₂ gas sensor according to the second embodiment.

FIG. 10A is a schematic planar pattern configuration diagram of the CO₂ gas sensor according to the present embodiment and FIG. 10B is a schematic cross-sectional structure diagram of the CO₂ gas sensor taken along line 18B-18B of FIG. 10A.

FIG. 11A is a schematic plane view of a wafer applied to the manufacturing of the CO₂ gas sensor according to the present embodiment and FIG. 11B is a schematic cross-sectional structure diagram taken along line 2B-2B of FIG. 11A.

FIG. 12A is a schematic plane view illustrating a process (first process) of the method of manufacturing a CO₂ gas sensor according to the present embodiment and FIG. 12B is a schematic cross-sectional structure diagram taken along line 3B-3B of FIG. 12A.

FIG. 13A is a schematic plane view illustrating a process (second process) of the method of manufacturing a CO₂ gas sensor according to the present embodiment and FIG. 13B is a schematic cross-sectional structure diagram taken along line 4B-4B of FIG. 13A.

FIG. 14A is a schematic plane view illustrating a process (third process) of the method of manufacturing a CO₂ gas sensor according to the present embodiment and FIG. 14B is a schematic cross-sectional structure diagram taken along line 5B-5B of FIG. 14A.

FIG. 15A is a schematic plane view illustrating a process (fourth process) of the method of manufacturing a CO₂ gas sensor according to the present embodiment and FIG. 15B is a schematic cross-sectional structure diagram taken along line 6B-6B of FIG. 15A.

FIG. 16A is a schematic plane view illustrating a process (fifth process) of the method of manufacturing a CO₂ gas sensor according to the present embodiment and FIG. 16B is a schematic cross-sectional structure diagram taken along line 7B-7B of FIG. 16A.

FIG. 17A is a schematic plane view illustrating a process (sixth process) of the method of manufacturing a CO₂ gas sensor according to the present embodiment and FIG. 17B is a schematic cross-sectional structure diagram taken along line 20B-20B of FIG. 17A.

FIG. 18A is a schematic plane view illustrating a process (seventh process) of the method of manufacturing a CO₂ gas sensor according to the present embodiment and FIG. 18B is a schematic cross-sectional structure diagram taken along line 21B-21B of FIG. 18A.

FIG. 19A is a schematic plane view illustrating a process (eighth process) of the method of manufacturing a CO₂ gas sensor according to the present embodiment and FIG. 19B is a schematic cross-sectional structure diagram taken along line 22B-22B of FIG. 19A.

FIG. 20A is a schematic plane view illustrating a process (ninth process) of the method of manufacturing a CO₂ gas sensor according to the present embodiment and FIG. 20B is a schematic cross-sectional structure diagram taken along line 24B-24B of FIG. 20A.

FIG. 21A is a schematic plane view illustrating a process (tenth process) of the method of manufacturing a CO₂ gas sensor according to the present embodiment and FIG. 21B is a schematic cross-sectional structure diagram taken along line 25B-25B of FIG. 21A.

FIG. 22A is a schematic plane view illustrating a process (eleventh process) of the method of manufacturing a CO₂ gas sensor according to the present embodiment and FIG. 22B is a schematic cross-sectional structure diagram taken along line 26B-26B of FIG. 22A.

FIG. 23A is a schematic plane view illustrating a process (twelfth process) of the method of manufacturing a CO₂ gas sensor according to the present embodiment and FIG. 23B is a schematic cross-sectional structure diagram taken along line 27B-27B of FIG. 23A.

FIG. 24 is a schematic bird's-eye configuration (perspective) view illustrating a cover of a package that accommodates the CO₂ gas sensor according to the present embodiment.

FIG. 25 is a schematic bird's-eye configuration (perspective) view illustrating a main body of a package that accommodates the CO₂ gas sensor according to the present embodiment.

FIG. 26 is a schematic block diagram illustrating the CO₂ gas sensor according to the present embodiment.

FIG. 27 is a schematic block diagram of a sensor package on which the CO₂ gas sensor according to the present embodiment is mounted.

FIG. 28 is a schematic block diagram of a sensor network employing the CO₂ gas sensor according to the present embodiment.

### DETAILED DESCRIPTION

Embodiments of the present disclosure will now be described with reference to the drawings. Further, in the following description of the drawings, like or similar reference numerals are used for like or similar parts. However, it should be noted that the plane views, side views, bottom views, and cross-sectional views are schematic, and the relationships between thicknesses and planar dimensions of respective components, and the like are different from those of reality. Thus, specific thicknesses or dimensions should be determined in consideration of the following description. Also, it is understood that parts having different dimensional relationships or ratios are included among the drawings.

Further, the embodiments described below are presented to illustrate apparatuses or methods for embodying the technical concept of the present disclosure and are not intended to specify the materials, features, structures, arrangements, and the like of the components to those shown below. The embodiments may be variously modified within the scope of claims.

### [Basic Principle of Semiconductor Type Gas Sensor]

First, a basic principle of a semiconductor type gas sensor using tin oxide (SnO₂) will be described.

An electric conductivity of SnO₂ is changed depending on an ambient gas concentration. That is, when a SnO₂ grain heated to a temperature of hundreds of degrees C. is cleaned and exposed to the air, oxygen in the air is adsorbed to a surface of the SnO₂ grain to capture an electron on the surface of the SnO₂ grain, which enters a state where the electricity does not flow. Meanwhile, when a reducing gas is present therearound, the oxygen adsorbed to the surface of the SnO₂ grain reacts with the reducing gas so as to be removed or an electron of the SnO₂ grain is free to make electricity easy to flow.

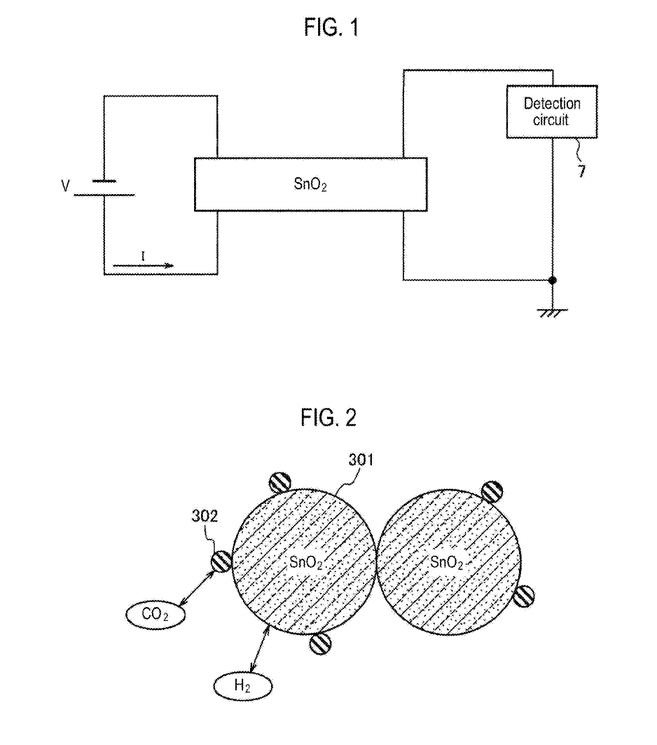

Based on a change in a resistance value, a detection circuit 7 (see FIG. 1) is configured to measure an ambient gas concentration.

### Comparative Example

A CO₂ gas sensor according to a comparative example is a semiconductor type gas sensor using the tin oxide (SnO₂), and as illustrated in FIG. 2, it is formed by adding lanthanum (La) 302 to a tin oxide 301. The lanthanum 302 is known to have high reactivity with CO₂, while the tin oxide 301 has a problem that reacts to various gases such as H₂ and CO. Thus, it is required to further enhance the selectivity of the CO₂ gas.

### First Embodiment

A first embodiment of the present disclosure will now be described. Further, in the following description, SnO₂ is tin oxide serving as a material of a gas-sensitive body, CO₂ is carbon dioxide serving as a gas to be measured, and La₂O₃ is lanthanum oxide serving as a rare earth oxide.

### (CO₂ Gas Sensor)

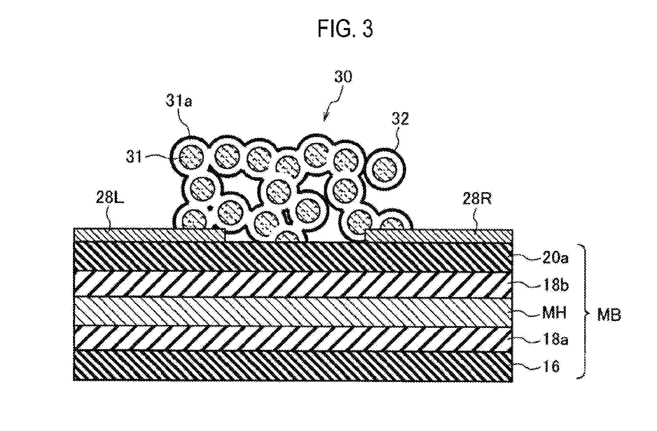

A schematic structure of a CO₂ gas sensor according to the first embodiment is illustrated in FIG. 3. As illustrated in FIG. 3, the CO₂ gas sensor according to the first embodiment is a semiconductor type gas sensor for detecting a CO₂ gas, and includes a gas-sensitive body 30 in which a surface of the tin oxide is coated with a thin film of a rare earth oxide, a pair of positive and negative electrodes 28L and 28R tightly formed on the gas-sensitive body 30, and a micro-heater MH for heating the gas-sensitive body 30. A membrane MB has a structure in which the micro-heater MH is embedded between SiO₂ films (insulating films) 18a and 18b and SiN films (protective films) 16 and 20a.

Specifically, as illustrated in FIG. 3, the surface of the SnO₂ grain 31 is entirely coated with a La₂O₃ film 32. Details thereof will be described later, but when the SnO₂ grain 31 is heated by the micro-heater MH at a temperature of hundreds of degrees C. a depletion layer 31a is formed in the SnO₂ grain 31. The La₂O₃ film 32 is an insulator, but since the electrodes 28L and 28R are connected by the SnO₂ grain 31 as a semiconductor, when a voltage is applied between the electrodes 28L and 28R, the current flows through the SnO₂ grain 31. Using a change in a resistance value of the gas-sensitive body 30, the detection circuit 7 (see FIG. 1) is configured to detect a CO₂ gas.

A particle diameter of the SnO₂ grain 31 is, for example, 1 to several tens pm. A width of the depletion layer 31a of the SnO₂ grain 31 is, for example, 5 to 10 nm. A thickness of the La₂O₃ film 32 is, for example, about 30 nm. As the particle diameter of the SnO₂ grain 31 is smaller, the sensitivity (a change in a resistance value) to a gas is increased.

With this configuration, since SnO₂ does not appear on a surface, SnO₂ is not in contact with a gas, and as a result, a reaction to a gas such as H₂ or CO is eliminated. Further, since the surface is entirely coated with La₂O₃, a CO₂ adsorption site is increased to enhance the sensitivity to the CO₂ gas.

### (Principle of Detecting CO₂ Gas)

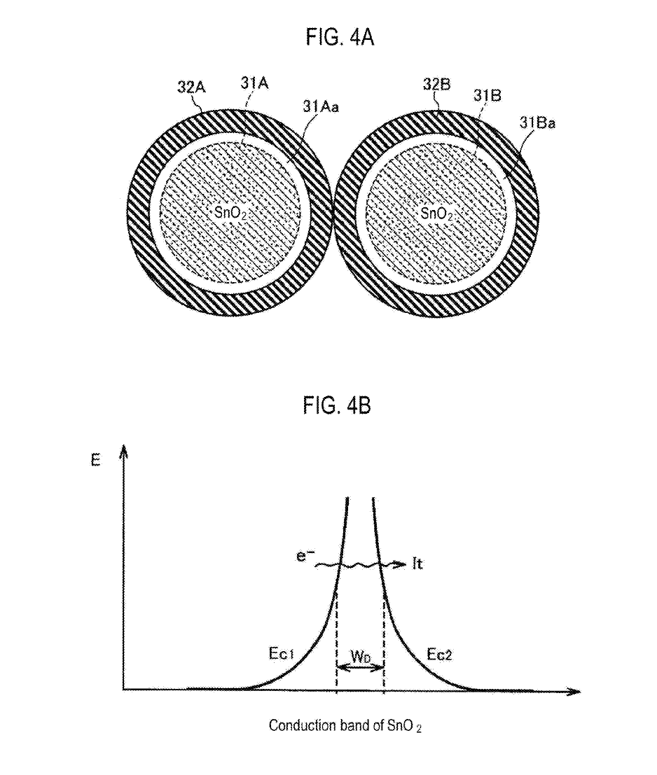

FIG. 4A and 4B are explanatory views illustrating a principle of detecting a CO₂ gas by the CO₂ gas sensor illustrated in FIG. 3, in which FIG. 4A is a schematic structure diagram of two adjacent SnO₂ grains 31A and 31B and FIG. 4B is a graph schematically illustrating conduction bands E_{c1} and E_{c2} of the SnO₂ grains 31A and 31B.

As described above, when the SnO₂ grains 31A and 31B are heated to a temperature of hundreds of degrees C. oxygen in the air captures electrons of the SnO₂ grains 31A and 31B and is adsorbed to the surface of the SnO₂ grains 31A and 31B. As a result, depletion layers 31Aa and 31Ba are formed in the SnO₂ grains 31A and 31B. The depletion layers 31Aa and 31Ba are electrically insulated regions without little electrons and a width W_{D} of the depletion layers 31Aa and 31Ba is 5 to 10 nm. Thus, when a high voltage is applied between the electrodes 28L and 28R, the electrons pass through the depletion layers 31Aa and 31Ba due to a tunneling effect to cause a current It to flow. Of course, as the width W_{D} of the depletion layers 31Aa and 31Ba is reduced, a resistance value is reduced, and as the width W_{D} of the depletion layers 31Aa and 31Ba is increased, a resistance value is increased.

Here, La₂O₃ is known to have high reactivity with CO₂. When a CO₂ gas is adsorbed to the La₂O₃ films 32A and 32B, the width W_{D} of the depletion layers 31Aa and 31Ba is reduced and a resistance value is reduced. Thus, when the surfaces of the SnO₂ grains 31A and 31B are entirely coated with the La₂O₃ films 32A and 32B, since SnO₂ does not appear on the surfaces, it is possible to enhance the sensitivity to the CO₂ gas.

The reason why the width W_{D} of the depletion layers 31Aa and 31Ba is reduced when the CO₂ gas is adsorbed to the La₂O₃ films 32A and 32B is thought as below. It is known in the related art that, when La₂O₃ is used in a p type silicon semiconductor, the capacitance is changed by a width of depletion layers when CO₂ is adsorbed to La₂O₃. Specifically, when CO₂ is adsorbed to La₂O₃, the capacitance is lowered and the width of the depletion layers is increased. SnO₂ used in the present embodiment is an n type semiconductor, and since P type and N type are in an inverse relation, it is considered that, when the CO₂ gas is adsorbed to the La₂O₃ films 32A and 32B, the width W_{D} of the depletion layers 31Aa and 31Ba is reduced.

### (Manufacturing Method)

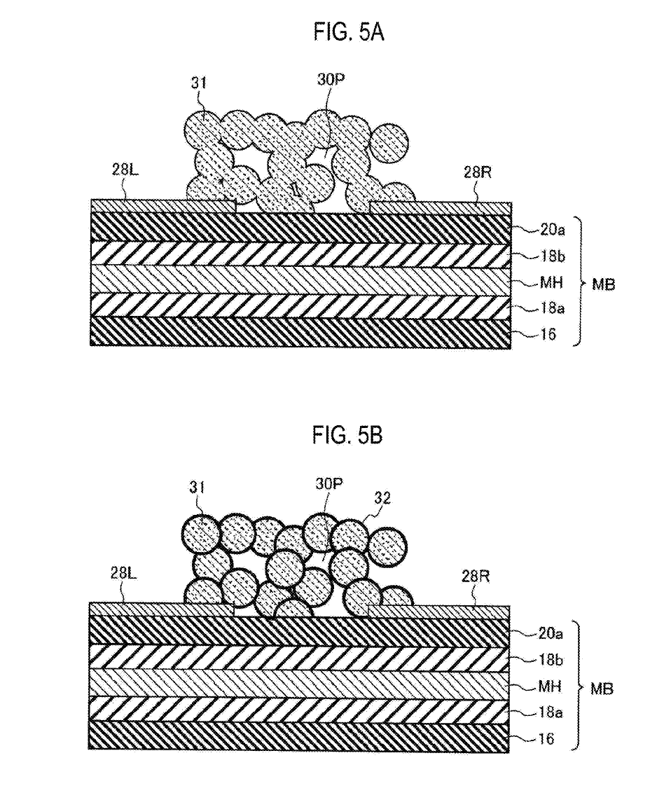

FIGS. 5A and 5B illustrate a method of manufacturing a CO₂ gas sensor according to the first embodiment. Here, in particular, a method of manufacturing a gas-sensitive body 30 having a porous SnO₂ structure will be described in detail.

First, as illustrated in FIG. 5A, Pt/Cr is printed on a membrane MB to form a pair of positive and negative electrodes 28L and 28R. A SnO₂—SiO₂ mixture film is formed on the electrodes 28L and 28R through sputtering, printing/sintering, a sol-gel method, or the like, and etched with a hydrogen fluoride-based solution to remove SiO₂. In FIGS. 5A and 5B, reference numeral 30P denotes an area where the SiO₂ was present.

Subsequently, as illustrated in FIG. 5B, the entire surface is coated with a La₂O₃ film 32 through atomic layer deposition (ALD), or the like. In some embodiments, a thickness of the La₂O₃ film 32 may be, for example, about 30nm. According to ALD, the entire surface may be uniformly coated with the La₂O₃ film 32, and further, a film thickness of the La₂O₃ film 32 may be adjusted in units of about 0.1 nm by adjusting a cycle number.

Through the aforementioned process, it is possible to manufacture the porous SnO₂ structure in which the surface of the SnO₂ grains 31 is uniformly entirely coated with the La₂O₃ film 32. As can be seen from FIGS. 5A and 5B, the electrodes 28L and 28R are connected by the SnO₂ grains 31 as a semiconductor. It is also possible to package a sensor device having such a porous SnO₂ structure.

### (Modifications of Electrode Layout)

Next, modifications of an electrode layout of the CO₂ gas sensor according to the first embodiment will be described.

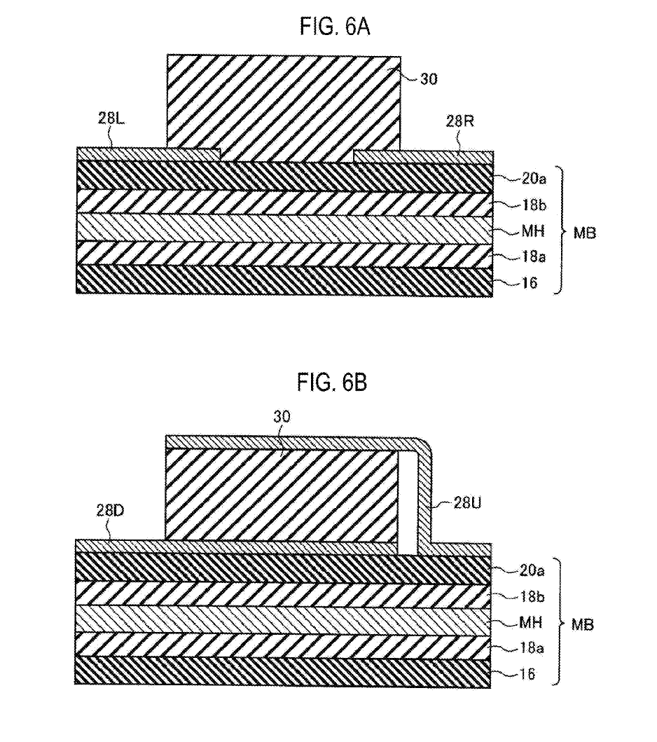

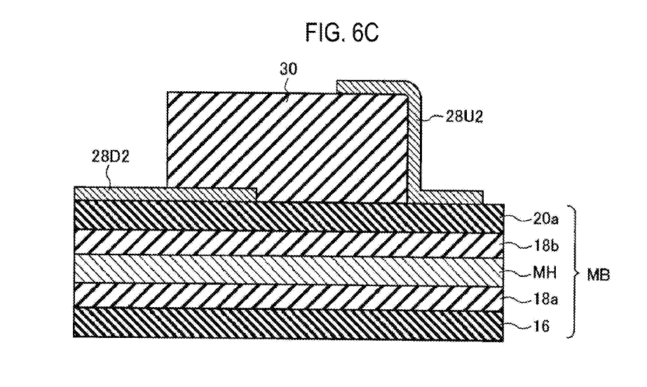

FIG. 6A illustrates the same electrode layout as that of FIG. 3. That is to say, a pair of positive and negative electrodes 28L and 28R is patterned on the same plane and a gas-sensitive body 30 is disposed to cover between the electrodes 28L and 28R.

FIG. 6B illustrates modification 1 of the electrode layout illustrated in FIG. 3. As illustrated in FIG. 6B, a gas-sensitive body 30 may be disposed between an electrode 28D as a lower electrode and an electrode 28U as an upper electrode.

FIG. 6C illustrates modification 2 of the electrode layout illustrated in FIG. 3. As illustrated in FIG. 6C, a portion of a lower surface of the gas-sensitive body 30 may be disposed on an electrode 28D2 as a lower electrode, and an electrode 28U2 as an upper electrode may be disposed in a portion of an upper surface of the gas-sensitive body 30.

As described above, according to the first embodiment, it is possible to realize the CO₂ gas sensor of the La₂O₃-coated SnO₂ base. Specifically, the surface of the SnO₂ grain 31 is entirely coated with the La₂O₃ film 32. With this configuration, since SnO₂ does not appear on the surface, it is possible to further enhance the selectivity of the CO₂ gas, compared with the comparative example. As a result, since no filter is required to remove a gas such as H₂ or CO, there is also an effect that may be easily miniaturized.

Further, here, although lanthanum oxide (La₂O₃) is illustrated as a rare earth oxide, instead of La₂O₃, a gadolinium oxide (Gd₂O₃) may also be used. Since Gd₂O₃ also has high reactivity with CO₂, the same effect can be obtained.

### Second Embodiment

Hereinafter, only differences of a second embodiment from the first embodiment will be described.

### (CO₂ Gas Sensor)

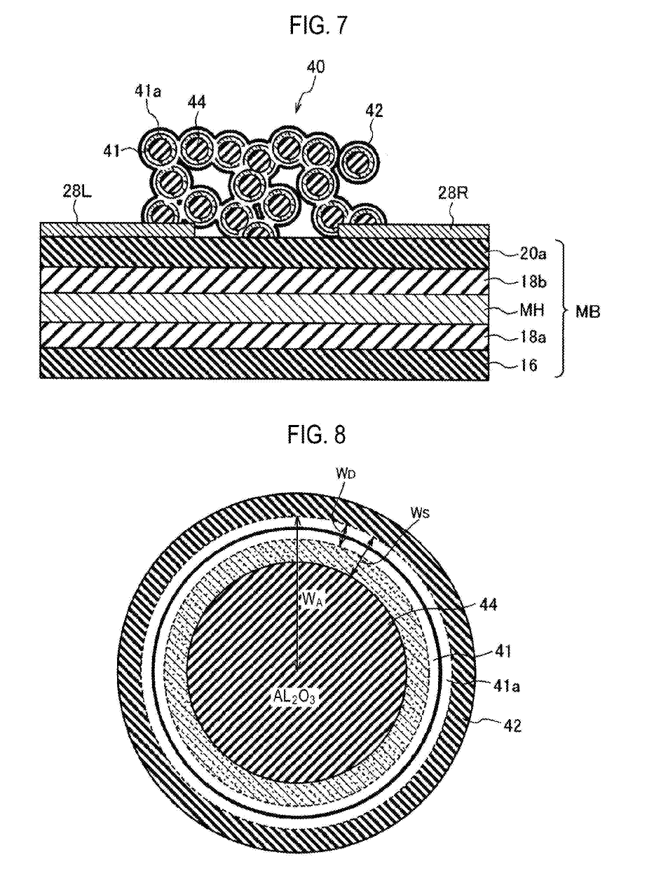

A schematic structure of a CO₂ gas sensor according to the second embodiment of the present disclosure is illustrated in FIG. 7. As illustrated in FIG. 7, the CO₂ gas sensor is the same as that of the first embodiment, except for a structure of a gas-sensitive body 40.

Specifically, as illustrated in FIG. 7, a surface of an aluminum oxide (Al₂O₃) grain 44 is entirely coated with a SnO₂ film 41 and a surface of the SnO₂ film 41 is entirely coated with a La₂O₃ film 42. Both the La₂O₃ film 42 and Al₂O₃ grain 44 are insulators. However, since the electrodes 28L and 28R are connected by the SnO₂ film 41 as a semiconductor, when a voltage is applied between the electrodes 28L and 28R, the current may flow through the SnO₂ film 41.

Also with this configuration, since SnO₂ does not appear on the surface, SnO₂ is not in contact with a gas, and as a result, there is no reaction to a gas such as H₂ or CO, as in the first embodiment. In addition, finally, since the surface is entirely coated with La₂O₃, a CO₂ adsorption site is increased to enhance the sensitivity to a CO₂ gas.

### (Principle of Detecting CO₂ Gas)

FIG. 8 is an explanatory view illustrating a principle of detecting a CO₂ gas by the CO₂ gas sensor illustrated in FIG. 7. The basic principle is the same as that of the first embodiment.

In the second embodiment, SnO₂ is formed as a thin film to increase a variation of a width of a depletion layer, compared with the first embodiment. That is, as illustrated in FIG. 8, when a width of the SnO₂ film 41 is Ws, a variation of the width W_{D} of the depletion layer 41a may be expressed as ΔW_{D}/W_{S}. Meanwhile, when a width obtained by adding a particle diameter of the Al₂O₃ grain 44 to the width of the SnO₂ film 4l is W_{A}, a variation of the width W_{D} of the depletion layer 41a may be expressed as ΔW_{D}/W_{A}. Since W_{S} is small relative to W_{A}, ΔW_{D}/Ws is increased relative to the case of ΔW_{D}/W_{A}. ΔW_{D}/Ws is equivalent to a variation of the width W_{D} of the depletion layer 4la in the second embodiment, and ΔW_{D}/W_{A} is equivalent to a variation of the width W_{D} of the depletion layer 4la in the first embodiment. Thus, it can be seen that, in the second embodiment, a variation in the width of the depletion layer is increased, compared with the first embodiment.

### (Manufacturing Method)

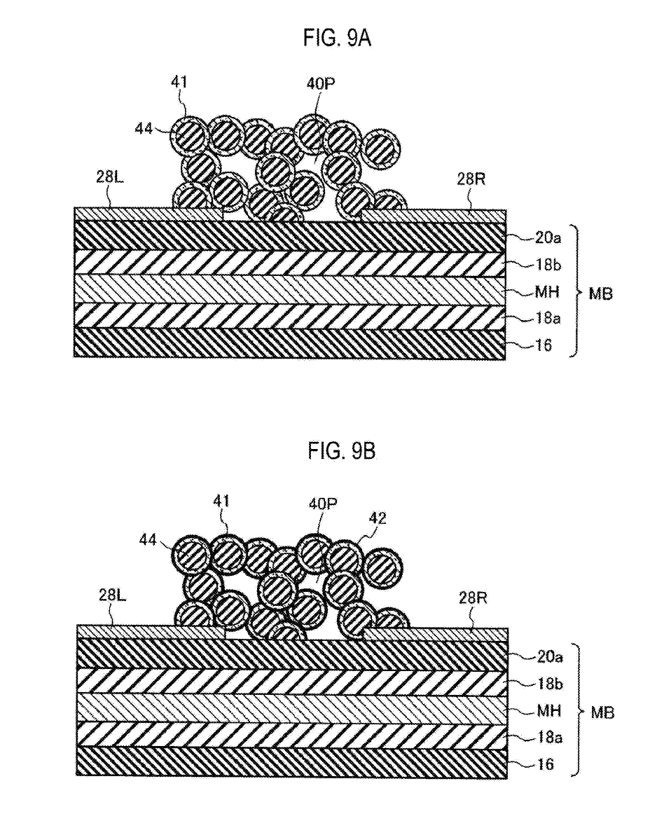

FIGS. 9A and 9B illustrate a method of manufacturing a CO₂ gas sensor according to the second embodiment. Here, in particular, a method of manufacturing a gas-sensitive body 40 having a porous SnO₂ structure will be described in detail.

First, as illustrated in FIG. 9A, Pt/Cr is printed on a membrane MB to form a pair of positive and negative electrodes 28L and 28R. An Al₂O₃—SiO₂ mixture film is formed on the electrodes 28L and 28R through sputtering, printing/sintering, a sol-gel method, or the like, and etched with a hydrogen fluoride-based solution to remove SiO₂. In FIGS. 9A and 9B, reference numeral 40P denotes an area where the SiO₂ was present.

Subsequently, the entire surface is uniformly coated with a SnO₂ film 4l through ALD or the like.

Further, as illustrated in FIG. 9B, the entire surface is uniformly coated with a La₂O₃ film 42 through ALD or the like. In some embodiments, a thickness of the La₂O₃ film 42 may be, for example, about 30 nm, as in the first embodiment.

Through the aforementioned process, it is possible to manufacture the porous SnO₂ structure in which the surface of the Al₂O₃ grains 44 is uniformly entirely coated with the SnO₂ film 4l and the surface of the SnO₂ film 4l is uniformly entirely coated with the La₂O₃ film 42. As can be seen from FIGS. 9A and 9B, the electrodes 28L and 28R are connected by the SnO₂ film 4l as a semiconductor. It is also possible to package a sensor device having such a porous SnO₂ structure.

As described above, in the second embodiment, the surface of the Al₂O₃ grain 44 is entirely coated with the SnO₂ film 4l and the surface of the SnO₂ film 4l is entirely coated with the La₂O₃ film 42. Accordingly, since the SnO₂ is formed as a thin film to increase a variation in the width of the depletion layer, it is possible to enhance the sensitivity to a CO₂ gas, compared with the first embodiment.

### [Specific Example of Device Structure]

Next, a specific example of the CO₂ gas sensor according to each embodiment will be described. Hereinafter, the electrode layout shown in FIG. 6B is illustrated, but the electrode layouts shown in FIGS. 6A and 6C may also be employed. Needless to say, as far as the La₂O₃-coated SnO₂-based CO₂ gas sensor is concerned, the materials, features, structures, arrangements, and the like of other components are not limited to those mentioned below.

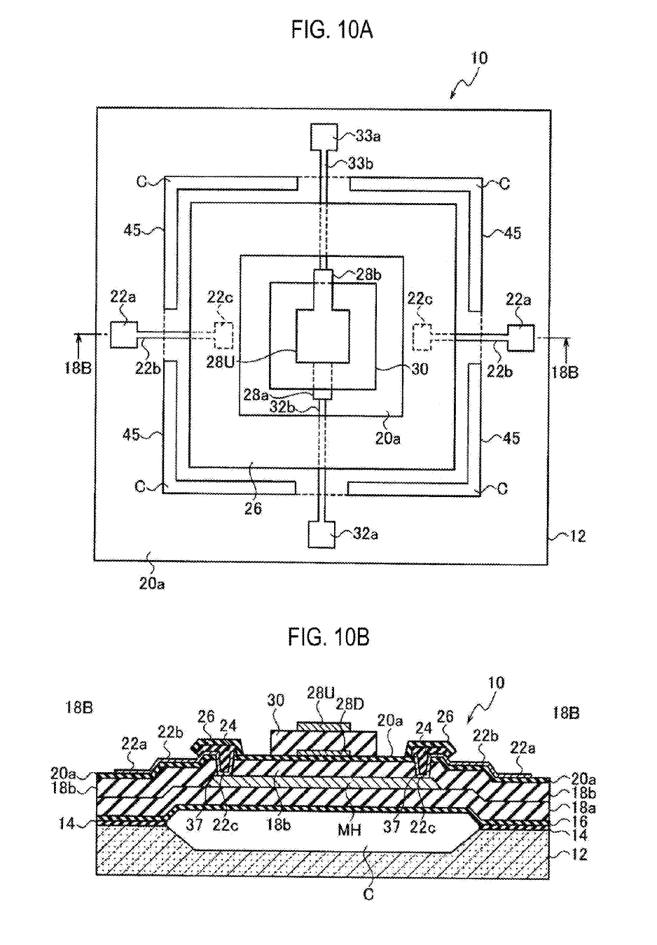

A schematic planar pattern configuration of the CO₂ gas sensor 10 according to each embodiment is illustrated in FIG. 10A, and a schematic cross-sectional structure of the sensor 10 having a micro-electro mechanical system (MEMS) beam structure taken along line 18B-18B of FIG. 10A is illustrated in FIG. lOB.

That is to say, as illustrated in FIGS. 10A and 10B, the CO₂ gas sensor 10 according to each embodiment includes a Si substrate 12 having an MEMS beam structure, a lower electrode (porous Pt/Ti film) 28D corresponding to a sensor part of a central portion and disposed on the Si substrate 12, a gas-sensitive body 30 disposed to cover the lower electrode 28D, and an upper electrode (Pt film) 28U disposed on the gas-sensitive body 30 facing the lower electrode 28D. First and second insulating layers (for example, a SiO₂ film) 18a and 18b are provided substantially on the entire surface of the Si substrate 12, and the lower electrode 28D is disposed on the second insulating layer 18b as an upper layer.

In the CO₂ gas sensor 10 according to each embodiment, a micro-heater MH is embedded between the first and second insulating layers 18a and 18b substantially corresponding to the sensor part. The micro-heater MH serves to heat the gas-sensitive body 30. For example, a predetermined voltage applied to heater connection pads 22a is supplied from heater electrode parts (Pt/Ti stacked films) 22c , which are formed along inner walls of openings 37 patterned on the second insulating layer 18b, through wiring parts 22b of a surface layer. In the heater electrode parts 22c, for example, the interior of the openings 37 is embedded by the SiO₂ film 24 and also coated by the SiN film 26 disposed to surround the sensor part. The heater connection pads 22a, the wiring parts 22b, and the heater electrode parts 22c are disposed in, for example, a direction along a cross-section of FIG. lOB.

Further, in the CO₂ gas sensor 10 according to each embodiment, for example, an electrode connection pad (detection terminal) 32a for applying a predetermined voltage to the lower electrode 28D, a wiring part 32b, one end of which is connected to the electrode connection pad 32a, an electrode connection pad 33a (detection terminal) for applying a predetermined voltage to the upper electrode 28U, and a wiring part 33b, one end of which is connected to the electrode connection pad 33a, are disposed on a surface layer in a direction perpendicular to the cross-section of FIG. 10B. The other end of the wiring part 32b of the electrode connection pad 32a is connected to a lead-out terminal 28a of the lower electrode 28D and the other end of the wiring part 33b of the electrode connection pad 33a is connected to a lead-out terminal 28b of the upper electrode 28U.

In addition, a detection circuit 7 for detecting a CO₂ gas is connected to the electrode connection pads 32a and 33a (for example, see FIG. 1).

In the CO₂ gas sensor 10 illustrated in FIG. 10A, the heater connection pads 22a are disposed on the left and right sides in a horizontal direction, and the electrode connection pad 32a and the electrode connection pad 33a are disposed on a lower end side and an upper end side in a vertical direction perpendicular to the horizontal direction, respectively. However, the positions of the electrode connection pads 32a and 33a may be exchanged or the positions of the heater connection pads 22a and the electrode connection pads 32a and 33a may be exchanged.

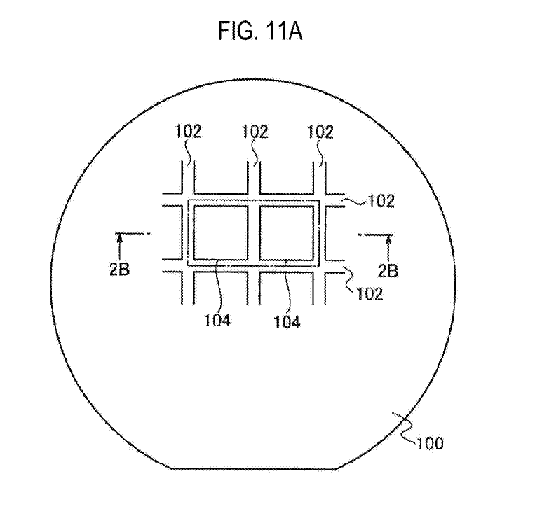

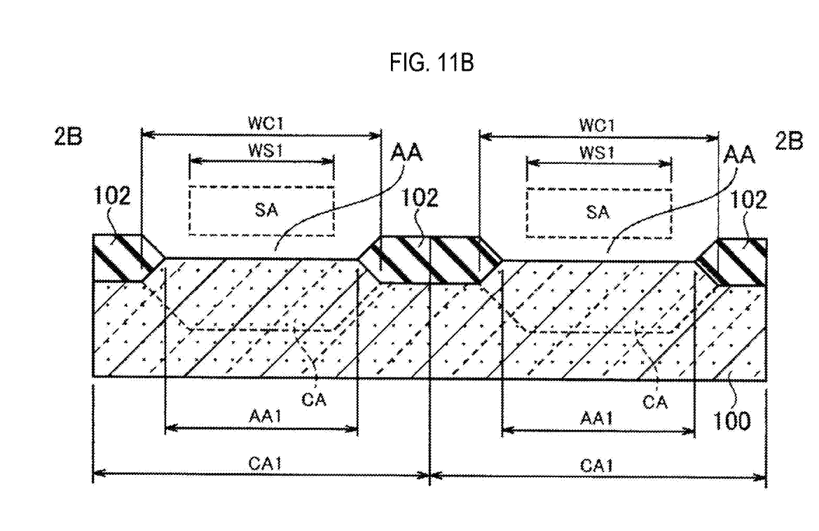

In the CO₂ gas sensor 10 according to each embodiment, a cavity part C having a vessel-shaped structure is formed as an MEMS beam structure on a surface portion of the Si substrate 12. That is to say, as illustrated in FIGS. 11A and 11B, the CO₂ gas sensor 10 according to each embodiment has a vessel-shaped MEMS beam structure in which the cavity part C is formed to have a vessel shape. The cavity part C substantially corresponds to, for example, an active area AA in a device area 104, which is defined by a device isolation area 102 on the wafer 100 capable of obtaining a plurality of Si substrates 12.

Here, a schematic planar configuration of the wafer 100 applied to manufacture the CO₂ gas sensor 10 according to each embodiment is illustrated in FIG. 11A and a schematic cross-sectional structure of the wafer 100 taken along line 2B-2B of FIG. 11A is illustrated in FIG. 11B.

As illustrated in FIGS. 11A and 11B, in the wafer 100, a plurality of device areas 104 are defined by the device isolation area 102 and diced along the device isolation area 102 at a final stage of a manufacturing process. Thus, the wafer 100 is divided into the plurality of Si substrates 12 to complete the gas sensor 10 in units of the Si substrate 12.

Further, in FIG. 11B, WCl indicates a width of a formation area CA of the cavity part C in a cross-sectional direction, WS1 indicates a width of a formation area SA of the sensor part in a cross-sectional direction, AA1 indicates a width of an active area AA in a cross-sectional direction, and CA1 indicates a width of the device area 104 in a cross-sectional direction.

Further, in the description of the present embodiment, Si represents silicon as a semiconductor material, Pt represents platinum as a porous material, and Ti represents titanium as an electrode material.

Here, the micro-heater MH is a polysilicon layer (polysilicon heater) having a thickness of, for example, 0.3 μm, to which boron (B) as a p type impurity is implanted with a high concentration through ion implantation. A resistance value of the micro-heater MH is about 300Ω. Further, the micro-heater MH may also be formed by a Pt heater or the like formed through printing. The micro-heater MH is formed to have substantially the same size as that of the sensor part.

The heater connection pads 22a, the wiring parts 22b, and the heater electrode parts 22c are formed by, for example, a stacked film (Pt/Ti stacked film) of a Ti film having a thickness of 20 nm and a Pt film having a thickness of 100 nm. The heater connection pads 22a and the wiring parts 22b are disposed on the SiN film 20a which covers the second insulating layer 18b.

The lower electrode 28D is formed with a thickness of, for example, about 100 nm, by a porous Pt/Ti film as a stacked film of a porous Pt film and a Ti film. The Ti film is used to cause the porous Pt film and the underlying SiN film 20a to be tightly bonded and more solidified.

The gas-sensitive body 30 has the tin oxide (SnO₂) as a main ingredient, and a surface of the tin oxide is coated with a thin film of a rare earth oxide. The gas-sensitive body 30 is interposed between the lower electrode 28D and the upper electrode 28U.

The Si substrate 12 having the MEMS beam structure has a thickness of, for example, about 10 μm, and is formed such that the cavity part C is substantially greater in size than the micro-heater MH to prevent an ambient heat from being released from the sensor part.

The MEMS beam structure may be an open structure in which the Si substrate 12 is disposed to surround the sensor part in a planar view. Further, the cavity part C may have a structure formed as the Si substrate 12 is bonded.

Further, the CO₂ gas sensor 10 according to each embodiment has the beam structure (vessel-shaped structure) with the MEMS structure, as a basic structure, thereby reducing the heat capacity of the sensor part and enhancing the sensor sensitivity.

Further, in the CO₂ gas sensor 10 according to each embodiment, the micro-heater MH is not limited to the case where the micro-heater MH is disposed between the first and second insulating layers 18a and 18b on the Si substrate 12 as the sensor part, and may be disposed below the Si substrate 12 or may be embedded within the Si substrate 12. Alternatively, it may be configured such that a stacked film (not shown) of a SiO₂ film/a SiN film including the micro-heater MH formed of polysilicon is formed on the surface of the Si substrate 12.

### (Manufacturing Method)

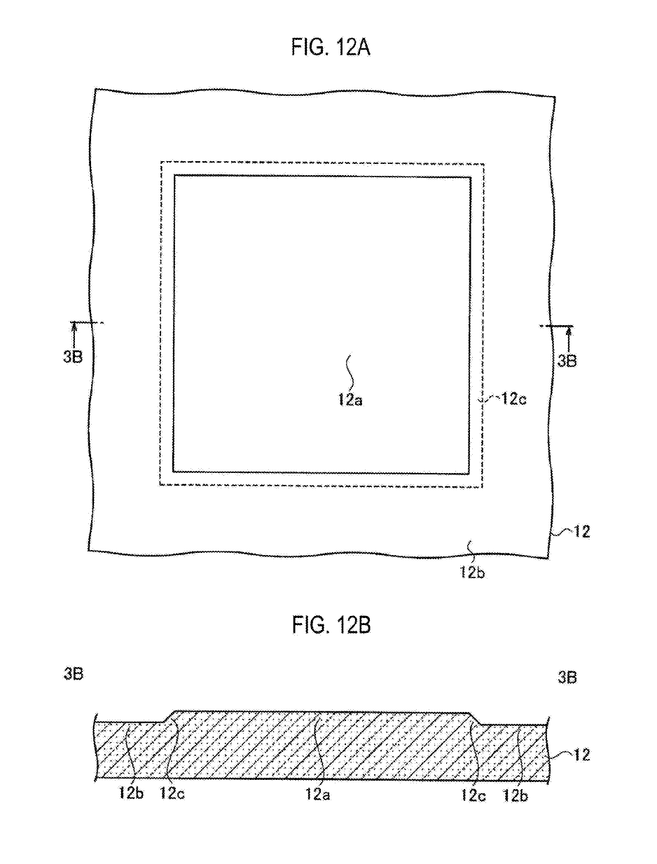

A method of manufacturing the CO₂ gas sensor 10 according to each embodiment illustrated in FIGS. 10A and 10B is illustrated in FIGS. 12A to 23B.

Originally, in the CO₂ gas sensor 10, a plurality of sensors 10 are collectively manufactured on the wafer 100, but here, a case where a sensor structure of the CO₂ gas sensor 10 is formed on the Si substrate 12 will be described for the convenience of description.

(a) First, as illustrated in FIGS. 12A and 12B, for example, an insulating film of the device isolation area 102 formed to have a grid shape is removed along a dicing line on the surface of the wafer 100 formed of Si and having a thickness of, for example, 10 μm, thereby forming an area 12a corresponding an active area AA and a non-active area 12b corresponding to other area, namely, the device isolation area 102, on the Si substrate 12. The area 12a corresponding to the active area AA has a shape with a sloped portion 12c in a peripheral portion, from a shape of the device isolation area 102.

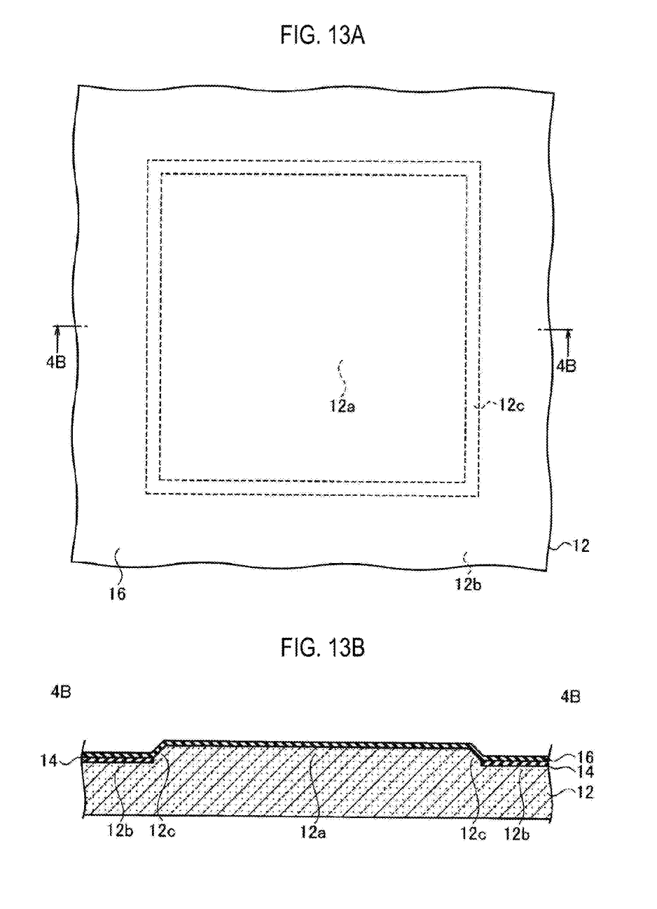

(b) Next, as illustrated in FIGS. 13A and 13B, a SiO₂ film having a thickness of about 0.5 μm is formed on an upper surface of the Si substrate 12 and the SiO₂ film on the sloped portion 12c and the area 12a corresponding to the active area AA is then selectively removed, thereby forming an insulating layer 14 formed of the SiO₂ film only in the non-active area 12b.

Subsequently, an insulating layer 16 formed of a SiON film and having a thickness of about 0.5 μm is uniformly formed on the upper surface of the Si substrate 12 through a plasma chemical vapor deposition (P-CVD) method or the like.

Alternatively, the insulating layer 14 may be formed by leaving a portion of the insulating film of the device isolation area 102.

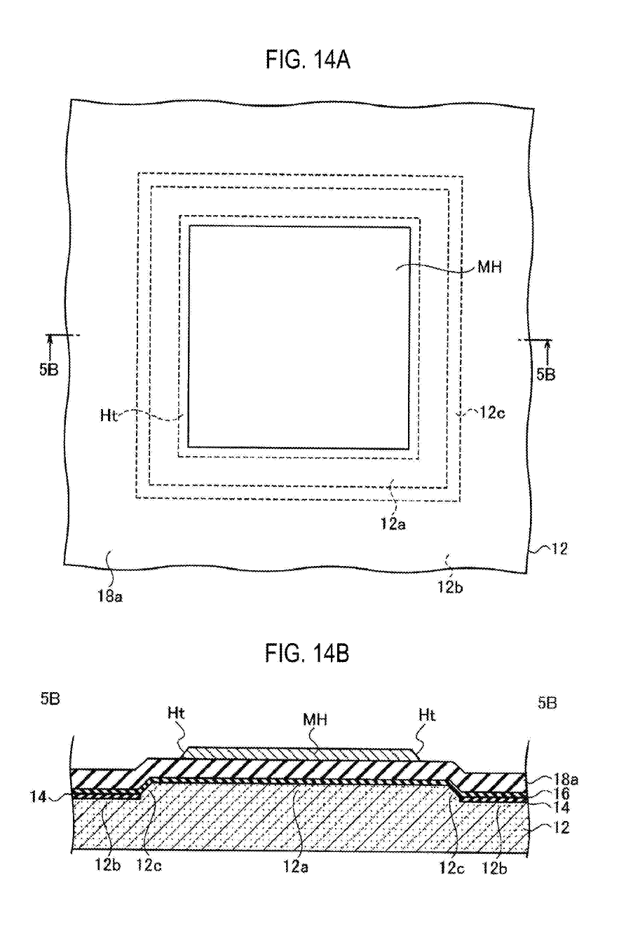

(c) Thereafter, as illustrated in FIGS. 14A and 14B, a first insulating layer 18a formed of the SiO₂ film and having a thickness of about 0.5 μm is formed on the insulating layer 16, and a polysilicon layer having a thickness of about 0.3 μm is then formed on an upper surface of the first insulating layer 18a. The polysilicon layer is patterned through etching or the like to form a micro-heater MH.

The micro-heater MH is formed to have a size (for example, about 300 μm²) almost equal to that of the sensor part on the area 12a corresponding to the active area AA. Further, B as a p type impurity is implanted with a high concentration to the micro-heater MH such that the micro-heater MH has a resistance value of 300Ω.

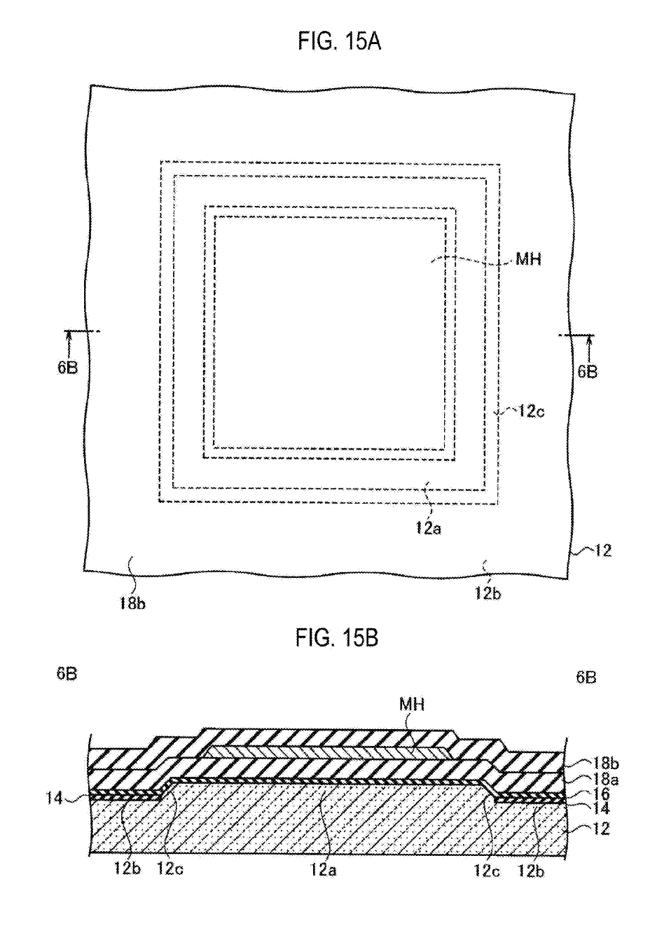

(d) Thereafter, as illustrated in FIGS. 15A and 15B, a SiON film (second insulating film) 18b having a thickness of about 0.5 μm is formed on the entire surface through a P-CVD method or the like.

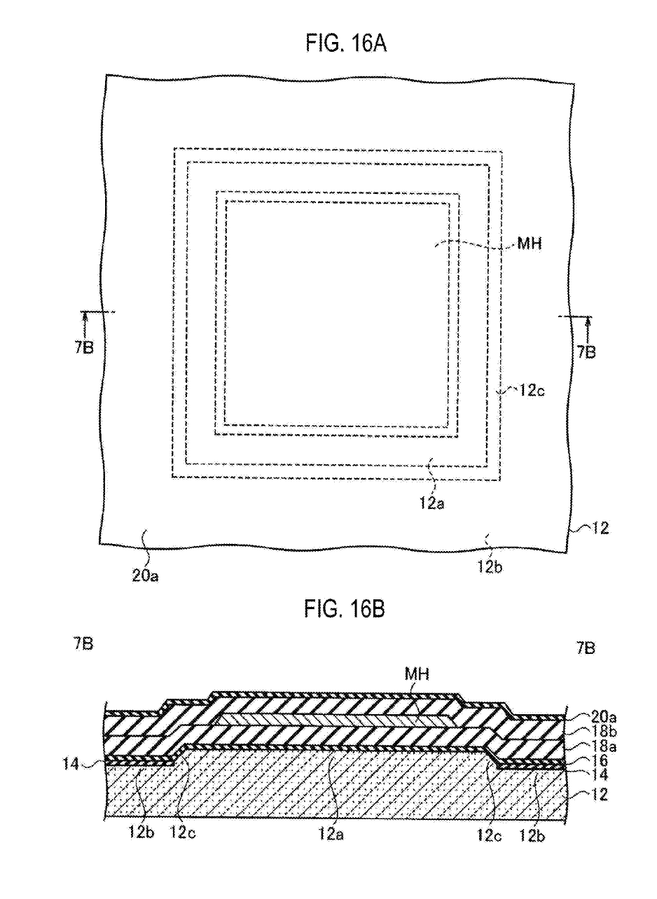

(e) Thereafter, as illustrated in FIGS. 16A and 16B, a SiN film (second insulating film) 20a having a thickness of about 0.5 μm is formed on the entire surface through the P-CVD method or the like.

A size of the cavity part C relies on a size of the CO₂ gas sensor 10 according to each embodiment. In some embodiments, the cavity part C may have a size of about 400 μm² so as to be substantially larger than the micro-heater MH. By forming the cavity part C to be substantially larger than the micro-heater MH, it becomes possible to simply suppress a heating by the micro-heater MH from being spread to a peripheral portion of the sensor part.

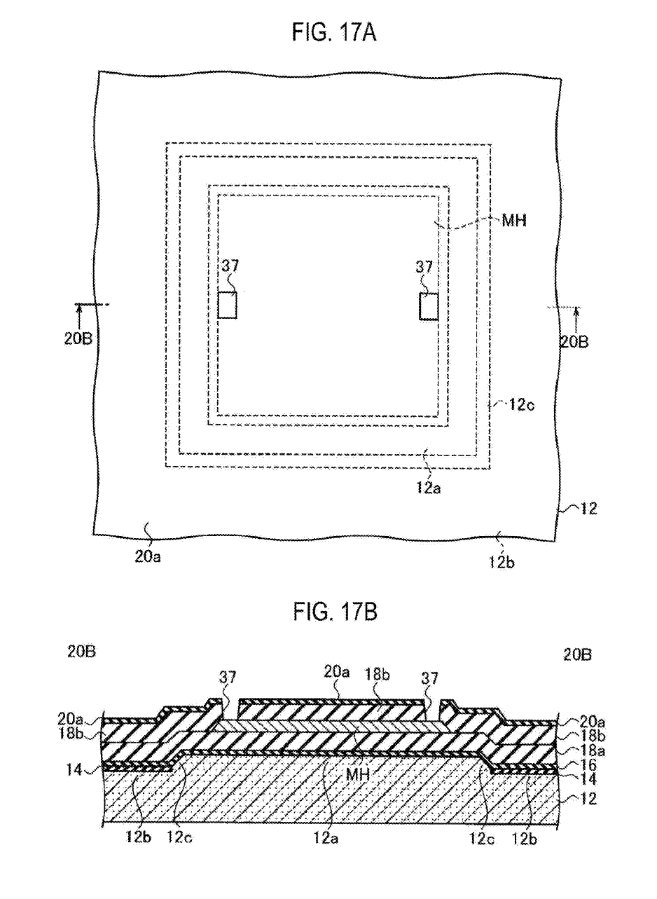

(f) Subsequently, as illustrated in FIGS. 17A and 17B, openings 37 for forming heater electrode parts 22c, leading to the micro-heater MH, are formed.

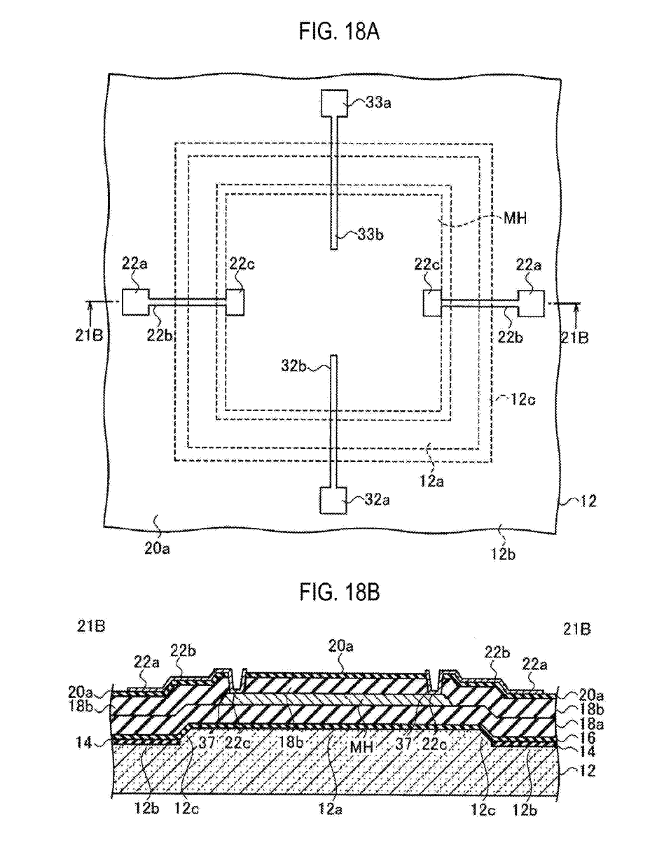

(g) Thereafter, as illustrated in FIGS. 18A and 18B, a Pt/Ti stacked film is deposited to have a thickness of about 0.5 μm. The Pt/Ti stacked film is patterned to form heater connection pads 22a , wiring parts 22b and heater electrode parts 22c.

Also, the Pt/Ti stacked film is patterned to form an electrode connection pad (detection terminal) 32a, a wiring part 32b, an electrode connection pad (detection terminal) 33a, and a wiring part 33b in a direction perpendicular to the heater connection pads 22a, the wiring parts 22b, and the heater electrode parts 22c.

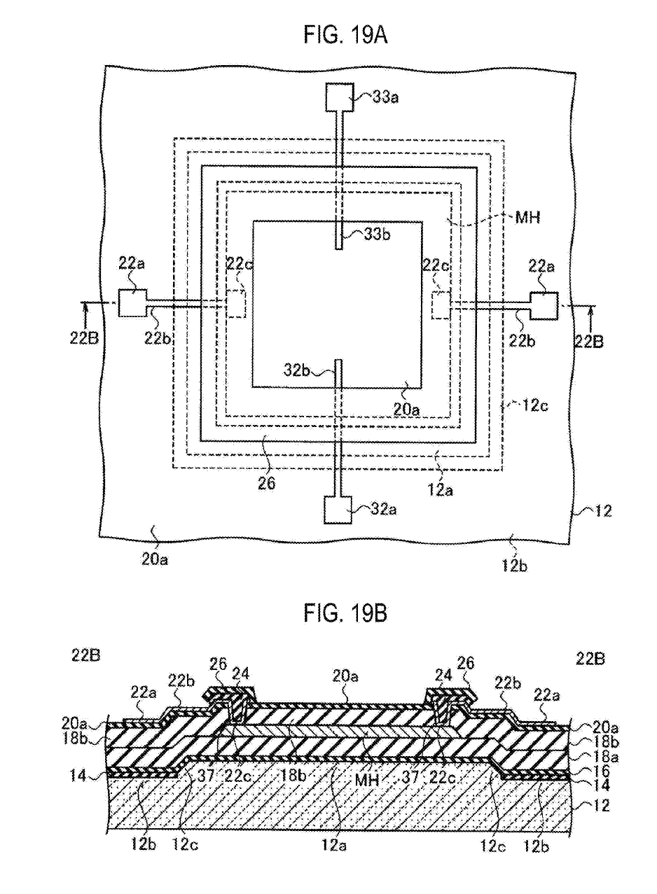

(h) Subsequently, as illustrated in FIGS. 19A and 19B, SiO₂ films 24 are formed to fill the inside of the openings 37 where the heater electrode parts 22c are formed along the inner wall of the openings 37, and SiN films 26 are formed. Then, for example, the SiO₂ films 24 and the SiN films 26 are patterned to surround the sensor part.

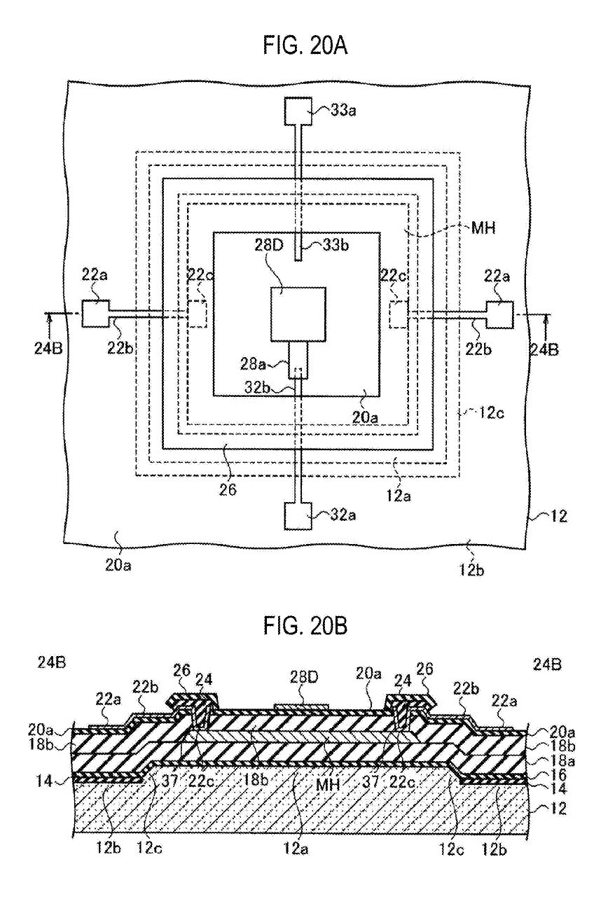

(i) Thereafter, as illustrated in FIGS. 20A and 20B, a lower electrode 28D formed of a Pt/Ti stacked film having a thickness of about 100 nm is formed on the SiN film 20a through a sputtering method or the like, and further, a lead-out terminal 28a of the lower electrode 28D, which extends from the sensor part, is connected to the wiring part 32b of the electrode connection pad 32a.

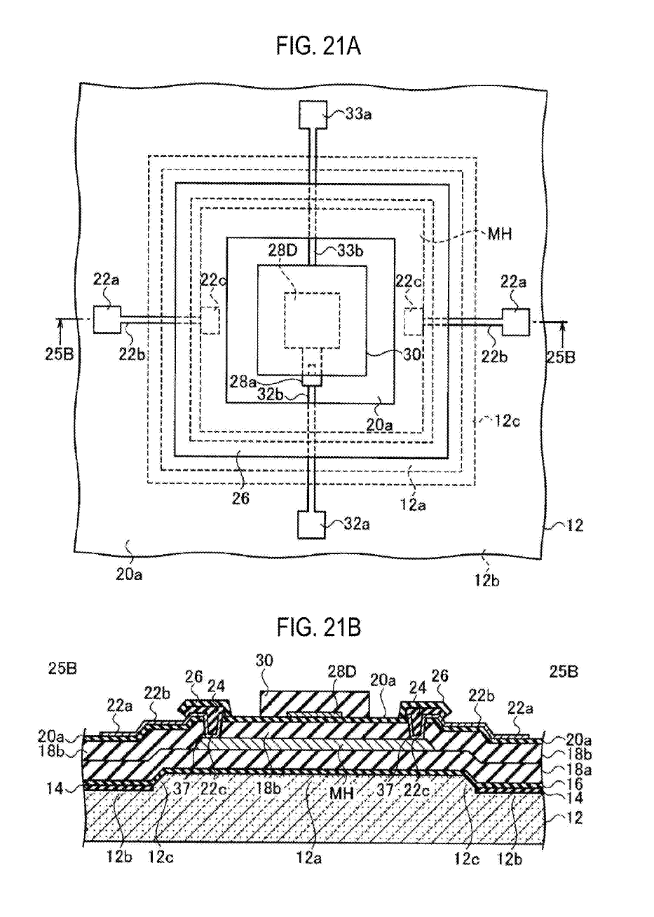

(j) Thereafter, as illustrated in FIGS. 21A and 21B, a gas-sensitive body 30 (see FIGS. 5A, 5B, 9A, and 9B) having a porous SnO₂ structure is formed to coat the lower electrode 28D. The gas-sensitive body 30 entirely coats the periphery of the lower electrode 28D, excluding the lead-out terminal 28a of the lower electrode 28D.

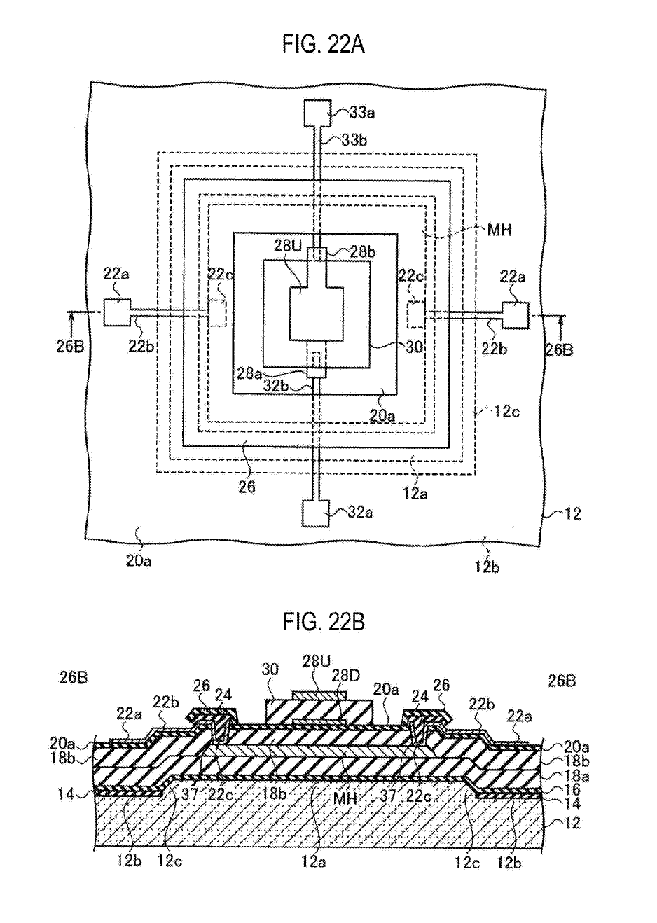

(k) Thereafter, as illustrated in FIGS. 22A and 22B, a Pt film having a thickness of about 100 nm is formed as the upper electrode 28U on a surface of the gas-sensitive body 30 in the sensor part facing the lower electrode 28D through a sputtering method, and further, the lead-out terminal 28b of the upper electrode 28U, which extends from the sensor part, is connected to the wiring part 33b of the electrode connection pad 33a.

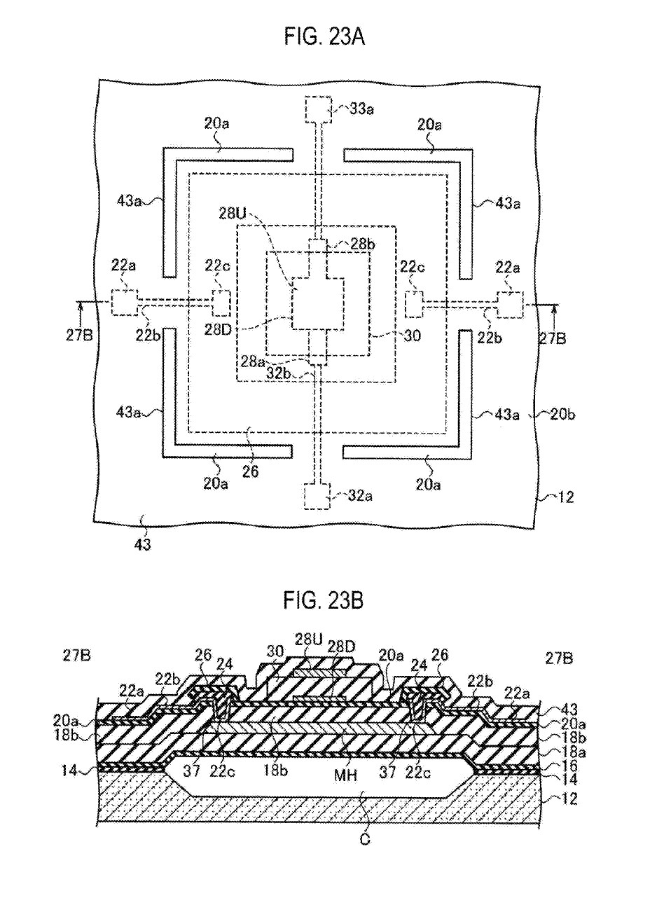

(l) Thereafter, as illustrated in FIGS. 23A and 23B, a protective SiO₂ film (mask) 43 having openings 43a for forming a cavity part C having a vessel-shaped structure as an MEMS beam structure is formed on the entire surface. Further, the Si substrate 12 of an area 12a corresponding to the active area AA is selectively depth-etched using the protective SiO₂ film 43 as a mask, thereby forming a cavity part C having a size of 400 μm² and having a vessel-shaped structure as the Si substrate 12 of the MEMS beam structure.

Finally, the protective SiO₂ film 43 is removed to obtain the CO₂ gas sensor 10 according to each embodiment having the configuration illustrated in FIGS. 10A and 10B.

As mentioned above, by forming the cavity part C to be substantially greater in size than the micro-heater MH, it is possible to simply suppress a heating by the micro-heater MH from being spread to the peripheral portion of the sensor part.

### (Package)

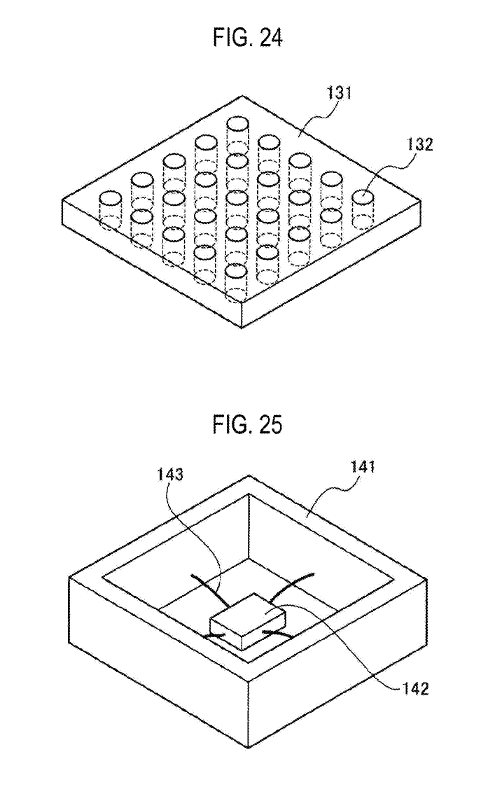

A schematic bird's-eye configuration illustrating a cover 131 of a package that accommodates the CO₂ gas sensor 10 according to each embodiment is illustrated in FIG. 24. As illustrated in FIG. 24, a plurality of through holes 132 for allowing a gas, but not a foreign object, to pass therethrough, are formed in the cover 131 of the package. A metal mesh, a small opening metal, a porous ceramic, or the like may be applied to the cover 131 of the package.

A schematic bird's-eye configuration illustrating a package body 141 that accommodates the CO₂ gas sensor 10 according to each embodiment is illustrated in FIG. 25. As illustrated in FIG. 25, a chip 142 of the CO₂ gas sensor 10 having a plurality of terminals is accommodated in the package body 141 and electrically connected to the package body 141 by a plurality of bonding wires 143. The cover 131 covers the package body 141 and the package body 141 is mounted on a print board or the like by soldering.

(Configuration Example of Sensor Node using Energy Harvester Power Source)

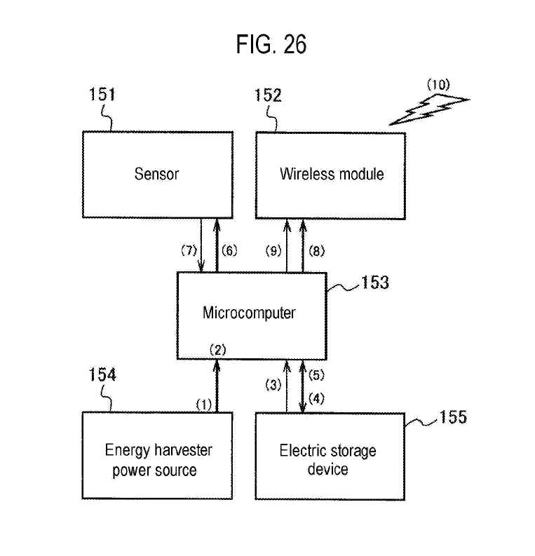

As illustrated in FIG. 26, the CO₂ gas sensor (sensor node) 10 according to each embodiment includes a sensor 151, a wireless module 152, a microcomputer 153, an energy harvester power source 154, and an electric storage device 155.

The sensor 151 has such a configuration as described in each embodiment.

The wireless module 152 is a module having an RF circuit and the like for transmitting and receiving wireless signals.

The microcomputer 153 has a function of managing the energy harvester power source 154 and applies an electric power from the energy harvester power source 154 to the sensor 151. Here, the microcomputer 153 may apply an electric power based on a heater electric power profile for saving power consumption in the sensor 151.

For example, the microcomputer 153 may apply a first electric power, which is a relatively large electric power, during a first period T1, and then apply a second electric power, which is a relatively small electric power, during a second period T2. Further, the microcomputer 153 may read data during the second period T2 and, after the second period T2 has lapsed, the microcomputer 153 may stop the application of electric power during a third period T3.

The energy harvester power source 154 obtains an electric power by harvesting energy such as sunlight or illumination light, or vibration or heat generated by a machine.

The electric storage device 155 is a lithium ion storage device or the like that can store an electric power.

An operation of such a sensor node will now be described.

First, as indicated by (1) of FIG. 26, an electric power is supplied from the energy harvester power source 154 to the microcomputer 153. Thus, the microcomputer 153 boosts (or steps up) a voltage from the energy harvester power source 154 as indicated by (2) of FIG. 26.

Next, after a voltage of the electric storage device 155 is read as indicated by (3) of FIG. 26, an electric power is supplied to the electric storage device 155 or an electric power is drawn from the electric storage device 155 as indicated by (4) and (5) of FIG. 26.

Thereafter, an electric power is applied to the sensor 151 based on the heater power profile as indicated by (6) of FIG. 26, and data such as a sensor resistance value, or a Pt resistance value is read as indicated by (7) of FIG. 26.

Thereafter, an electric power is supplied to the wireless module 152 as indicated by (8) of FIG. 26, and the data such as the sensor resistance value, or the Pt resistance value is transmitted to the wireless module 152 as indicated by (9) of FIG. 26.

Finally, as indicated by (10) of FIG. 10, the data such as the sensor resistance value, the Pt resistance value, or the like is wirelessly transmitted by the wireless module 152.

### (Sensor Package: Block Configuration)

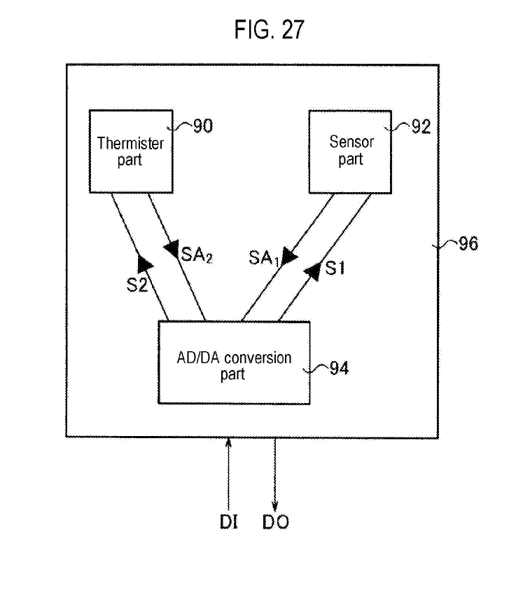

A schematic block configuration of the sensor package 96 including the CO₂ gas sensor 10 according to each embodiment is illustrated in FIG. 27.

As illustrated in FIG. 27, the sensor package 96 including the CO₂ gas sensor 10 according to each embodiment includes a thermister part 90 for temperature sensing, a sensor part 92 for a CO₂ gas sensing, an AD/DA conversion part 94 for receiving analog information SA₂ and SA₁ from the thermister part 90 and the sensor part 92 and transmitting control signals S2 and S1 to the thermister part 90 and the sensor part 92, and digital input/output signals DI and DO from the outside.

As the thermister part 90, for example, an NTC thermister, a PTC thermister, a ceramic PTC, a polymer PTC, a CTR thermister, or the like may be applied.

The CO₂ gas sensor 10 according to each embodiment may be applied to the sensor part 92.

### (Sensor Network)

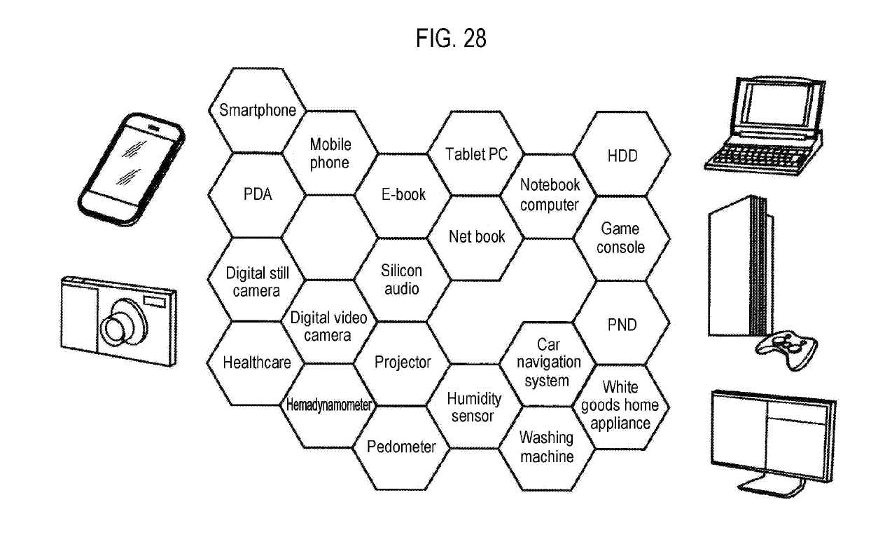

A schematic block configuration of a sensor network system employing the CO₂ gas sensor 10 according to each embodiment is illustrated in FIG. 28.

As illustrated in FIG. 28, the sensor network is a network formed by connecting a plurality of sensors to each other. A new attempt using the sensor network has already started in various fields such as plants, medical/health care, traffic, construction, agriculture, environment management, and the like.

In these fields, since it is necessary to use highly reliable sensors with high durability, it is desirable to apply the CO₂ gas sensor 10 according to each embodiment. This CO₂ gas sensor 10 has excellent selectivity of a CO₂ gas, which can provide a reliable sensor network.

As described above, the CO₂ gas sensor 10 according to the present embodiment is a semiconductor type gas sensor for detecting a CO₂ gas, and includes a gas-sensitive body 30 in which a surface of SnO₂ is coated with a thin film of a rare earth oxide, a pair of positive and negative electrodes 28L and 28R tightly formed on the gas-sensitive body 30, and a micro-heater MH for heating the gas-sensitive body 30. With this configuration, since SnO₂ does not appear on the surface, it is possible to further enhance the selectivity of the CO₂ gas.

Specifically, the surface of the SnO₂ grain 31 may be entirely coated with a La₂O₃ film 32. Thus, it is possible to reliably prevent the appearance of SnO₂ on the surface.

Further, the pair of positive and negative electrodes 28L and 28R may be electrically connected by the SnO₂ grain 31. Thus, when a voltage is applied between the electrodes 28L and 28R, current can flow through the SnOP₂ grain 31 as a semiconductor.

In addition, a surface of an Al₂O₃ grain 44 may be entirely coated with the SnO₂ film 41 and a surface of the SnO₂ film 41 may be entirely coated with a La₂O₃ film 42. With this configuration, since the SnO₂ may be formed as a thin film to increase a variation in a width of a depletion layer, it is possible to enhance the sensitivity to a CO₂ gas.

Furthermore, the pair of positive and negative electrodes 28L and 28R may be electrically connected by the SnO₂ film 41. Thus, when a voltage is applied between the electrodes 28L and 28R, current can flow through the SnO₂ film 41 as a semiconductor.

Moreover, the surface of SnO₂ may be uniformly entirely coated with the thin film of the rare earth oxide. Thus, it is possible to precisely detect a CO₂ gas.

Also, the rare earth oxide may be La₂O₃ or Gd₂O₃. Since La₂O₃ and Gd₂O₃ also have high reactivity with CO₂, it is possible to enhance the sensitivity to a CO₂ gas.

In addition, a detection circuit 7 for detecting a CO₂ gas using a change in a resistance value made in the gas-sensitive body 30 when a voltage is applied between the pair of positive and negative electrodes 28L and 28R may be provided. With this configuration, it is possible to easily detect a CO₂ gas based on a change in a resistance value.

A substrate 12 having a beam structure with an MEMS structure may be provided. The beam structure may be a vessel-shaped structure in which the cavity part C of a vessel shape is formed in the substrate 12. That is to say, employing the beam structure (vessel-shaped structure) having the MEMS structure as a basic structure, it is possible to reduce the heat capacity of the sensor part and enhance the sensor sensitivity.

Further, the cavity part C may be substantially greater in size than the micro-heater MH. Thus, it is possible to simply suppress a heating by the micro-heater MH from being spread to the peripheral portion of the sensor part.

The method of manufacturing a CO₂ gas sensor according to the present embodiment is a method of manufacturing a semiconductor type gas sensor for detecting a CO₂ gas, and includes a process of forming a micro-heater MH, a process of forming a pair of positive and negative electrodes 28L and 28R on the micro-heater MH, and a process of tightly forming a gas-sensitive body 30 in which a surface of SnO₂ is coated with a rare earth oxide thin film between the pair of positive and negative electrodes 28L and 28R. With this configuration, since SnO₂ does not appear on the surface, it is possible to further enhance the selectivity of a CO₂ gas.

Specifically, in the process of forming the gas-sensitive body 30, a surface of the SnO₂ grain 31 may be entirely coated with the La₂O₃ film 32 through an ALD method. Thus, it is possible to reliably prevent the appearance of SnO₂ on the surface.

The process of forming the gas-sensitive body 30 may be configured such that, a SnO₂—SiO₂ mixture film is formed on the pair of positive and negative electrodes 28L and 28R and etched with a hydrogen fluoride-based solution to remove SiO₂, and the entire surface is coated with the La₂O₃ film 32. Thus, it is possible to electrically connect the pair of positive and negative electrodes 28L and 28R by the SnO₂ grain 31.

The process of forming the gas-sensitive body 30 may configured such that, the surface of the Al₂O₃ grain 44 is entirely coated with the SnO₂ film 41 and the surface of the SnO₂ film 41 is entirely coated with the La₂O₃ film 42 through an ALD method. With this configuration, since the SnO₂ is formed as a thin film to increase a variation in a width of a depletion layer, it is possible to enhance the sensor sensitivity to a CO₂ gas.

The process of forming the gas-sensitive body 30 may configured such that, an Al₂O₃3-SiO₂ mixture film is formed on the pair of positive and negative electrodes 28L and 28R and etched with a hydrogen fluoride-based solution to remove SiO₂, the entire surface is coated with the SnO₂ film 41, and then the entire surface is coated with the La₂O₃ film 42. Thus, it is possible to electrically connect the pair of positive and negative electrodes 28L and 28R by the SnO₂ film 41.

In the process of forming the gas-sensitive body 30, SnO₂ may be uniformly entirely coated with a rare earth oxide thin film. Thus, it is possible to precisely detect a CO₂ gas.

In the process of forming the gas-sensitive body 30, La₂O₃ or Gd₂O₃ may be used as a rare earth oxide. Since La₂O₃ and Gd₂O₃ have high reactivity with CO₂, it is possible to enhance the sensitivity to a CO₂ gas.

The method may further include a process of forming a detection circuit for detecting a CO₂ gas using a change in a resistance value made in the gas-sensitive body 30 when a voltage is applied between the pair of positive and negative electrodes 28L and 28R. With this configuration, it is possible to easily detect a CO₂ gas based on a change in a resistance value.

The method may further include a process of forming the substrate 12 having a beam structure with an MEMS structure. In the process of forming the substrate 12, the cavity part C having a vessel shape, as a beam structure, may be formed in the substrate 12. That is, employing the beam structure (vessel-shaped structure) having the MEMS structure as a basic structure, it is possible to reduce the heat capacity of the sensor part and enhance the sensor sensitivity.

In the process of forming the substrate 12, the cavity part C may be formed to be substantially greater in size than the micro-heater MH. Thus, it is possible to simply suppress a heating by the micro-heater MH from being spread to the peripheral portion of the sensor part.

The sensor network system according to the present embodiment includes any one of the aforementioned CO₂ sensors, which can provide a high reliable sensor network.

As described above, it is possible to provide a semiconductor type gas sensor capable of further enhancing the selectivity of a CO₂ gas, a method of manufacturing a semiconductor type gas sensor, and a sensor network system.

### Other Embodiments

As mentioned above, although some embodiments have been described, the description and drawings constituting part of the present disclosure are merely illustrative and should not be understood to be limiting. Various alternative embodiments, examples, and operating techniques will be apparent to those skilled in the art from the present disclosure.

Thus, the present disclose includes a variety of embodiments and the like that are not disclosed herein.

The semiconductor type gas sensor according to the present embodiment can be applied to a CO₂ gas sensor. Further, the CO₂ gas sensor can be applied to an air cleaner or a sensor network.

According to some embodiments of the present disclosure in, it is possible to provide a semiconductor type gas sensor capable of further enhancing selectivity of a CO₂ gas, a method of manufacturing a semiconductor type gas sensor, and a sensor network system.

While certain embodiments have been described, these embodiments have been presented by way of example only, and are not intended to limit the scope of the disclosures. Indeed, the novel methods and apparatuses described herein may be embodied in a variety of other forms; furthermore, various omissions, substitutions and changes in the form of the embodiments described herein may be made without departing from the spirit of the disclosures. The accompanying claims and their equivalents are intended to cover such forms or modifications as would fall within the scope and spirit of the disclosures.

What is claimed is:

1. A semiconductor type gas sensor for detecting a CO₂ gas, the sensor comprising:
a gas-sensitive body in which a surface of a tin oxide is coated with a thin film of a rare earth oxide;
a pair of positive and negative electrodes tightly formed on the gas-sensitive body; and
a micro-heater configured to heat the gas-sensitive body.

2. The sensor of claim 1, wherein a surface of a tin oxide grain is entirely coated with the thin film of the rare earth oxide.

3. The sensor of claim 2, wherein the pair of positive and negative electrodes are electrically connected by the tin oxide grain.

4. The sensor of claim 1, wherein a surface of an aluminum oxide grain is entirely coated with a thin film of the tin oxide and a surface of the thin film of the tin oxide is entirely coated with the thin film of the rare earth oxide.

5. The sensor of claim 4, wherein the pair of positive and negative electrodes are electrically connected by the thin film of the tin oxide.

6. The sensor of claim 1, wherein the surface of the tin oxide is uniformly entirely coated with the thin film of the rare earth oxide.

7. The sensor of claim 1, wherein the rare earth oxide is a lanthanum oxide or a gadolinium oxide.

8. The sensor of claim 1, further comprising a detection circuit configured to detect a CO₂ gas using a change in a resistance value made in the gas-sensitive body when a voltage is applied between the pair of positive and negative electrodes.

9. The sensor of claim 1, further comprising a substrate having a beam structure with an MEMS structure,
wherein the beam structure has a vessel-shaped structure in which a cavity part of a vessel shape is formed in the substrate.

10. The sensor of claim 9, wherein the cavity part is substantially greater in size than the micro-heater.

11. A method of manufacturing a semiconductor type gas sensor for detecting a CO₂ gas, comprising:
forming a micro-heater;
forming a pair of positive and negative electrodes on the micro-heater; and
tightly forming a gas-sensitive body in which a surface of a tin oxide is coated with a thin film of a rare earth oxide, between the pair of positive and negative electrodes.

12. The method of claim 11, wherein the act of forming a gas-sensitive body includes:
coating an entire surface of a tin oxide grain with the thin film of the rare earth oxide through an atomic deposition method.

13. The method of claim 12, wherein the act of forming a gas-sensitive body includes:
forming a mixture film of the tin oxide and silicon oxide on the pair of positive and negative electrodes;
etching the mixture film with a hydrogen fluoride-based solution to remove the silicon oxide; and
coating an entire surface of the mixture film with the thin film of the rare earth oxide.

14. The method of claim 11, wherein the act of forming a gas-sensitive body includes:
coating an entire surface of an aluminum oxide grain with a thin film of the tin oxide through an atomic layer deposition method; and
coating an entire surface of the thin film of the tin oxide with the thin film of the rare earth oxide.

15. The method of claim 14, wherein the act of forming a gas-sensitive body includes:
forming a mixture film of aluminum oxide and silicon oxide on the pair of positive and negative electrodes;
etching the mixture film with a hydrogen fluoride-based solution to remove the silicon oxide;
coating an entire surface of the mixture film with the thin film of the tin oxide; and
coating the entire surface of the mixture film with the thin film of the rare earth oxide.

16. The method of claim 11, wherein the act of forming a gas-sensitive body includes:
uniformly coating an entire surface of the tin oxide with the thin film of the rare earth oxide through an atomic layer deposition method.

17. The method of claim 11, in the act of forming a gas-sensitive body, a lanthanum oxide or a gadolinium oxide is used as the rare earth oxide.

18. The method of claim 11, further comprising:
forming a detection circuit for detecting a CO₂ gas using a change in a resistance value made in the gas-sensitive body when a voltage is applied between the pair of positive and negative electrodes.

19. The method of claim 11, further comprising:
forming a substrate having a beam structure with an MEMS structure,
wherein the act of forming a substrate includes forming a cavity part of a vessel shape as the beam structure in the substrate.

20. The method of claim 19, wherein the act of forming a substrate includes forming the cavity part substantially greater in size than the micro-heater.

21. A sensor network system comprising the semiconductor type gas sensor of claim 1.
